(19) United States
(12) Patent Application Publication
(10) Pub. No.: US 2026/0043071 A1
(43) Pub. Date: Feb. 12, 2026

(54) IMMOBILIZED CRISPR ENRICHES CAPTURED TARGET PATHOGENS (ICECAP) ASSAY CHEMISTRY KEY DIFFERENTIATORS

(71) Applicant: MRIGlobal, Kansas City, MO (US)

(72) Inventors: Julie Lucas, Silver Spring, MD (US); Chelsey Smith, Arlington, VA (US); Richard Winegar, Palm Bay, FL (US)

(21) Appl. No.: 18/736,480

(22) Filed: Jun. 6, 2024

Related U.S. Application Data
(60) Provisional application No. 63/506,594, filed on Jun. 7, 2023.

(51) Int. Cl. C12Q 1/6837

(52) U.S. Cl. CPC C12Q 1/6837

(57) ABSTRACT

The present disclosure relates to an assay method for detection of target nucleic acid. The method includes immobilizing a guide RNA (gRNA) to an immobilization surface; complexing a Cas enzyme to the gRNA; adding a target nucleic acid; labeling the target nucleic acid; detecting the target nucleic acid without amplification; and determining one or more results based on the detected target nucleic acid via a reader.

### CROSS-REFERENCE TO RELATED APPLICATION

This application claims priority to and the benefit of U.S. Provisional Patent App. Ser. No. 63/506,594 filed on Jun. 7, 2023, entitled “IMMOBILIZED CRISPR ENRICHES CAPTURED TARGET PATHOGENS (ICECAP) ASSAY CHEMISTRY KEY DIFFERENTIATORS.” The content of the aforementioned patent application is hereby expressly incorporated by reference in its entirety for all purposes.

### STATEMENT REGARDING FEDERALLY SPONSORED RESEARCH OR DEVELOPMENT

This invention was made with government support under grant number BTO N6600121C4048, awarded by the DARPA. The government has certain rights in the invention.

### REFERENCE TO SEQUENCE LISTING

The instant application contains a Sequence Listing that has been submitted electronically in .XML format and is hereby incorporated by reference in its entirety. The Sequence Listing: is named 11202-190US, is 4,978 bytes in size, and was created on Jun. 6, 2024.

### BACKGROUND

Current biosurveillance and diagnostic systems aim to detect a broad range of biothreats and pathogens of interest in a timely and user-friendly manner. Traditional PCR-based assays typically take weeks to months to develop, validate, and incorporate for use on devices that can detect relatively small panels of targets.

### BRIEF SUMMARY OF THE DISCLOSURE

According to a first aspect, the present disclosure includes an assay method for detection of target nucleic acid, the method comprising: immobilizing a guide RNA (gRNA) to an immobilization surface; complexing a Cas enzyme to the gRNA; adding a target nucleic acid; labeling the target nucleic acid; detecting the target nucleic acid without amplification; and determining one or more results based on the detected target nucleic acid via a reader.

In a second aspect of the assay method of the first aspect or any other aspect, wherein: the target nucleic acid is labeled with a Cas sandwich protein comprising a randomized gRNA library complexed with the Cas sandwich protein; the randomized gRNA library comprises a plurality of secondary gRNAs; and the plurality of secondary gRNAs are labeled and comprise a tail complexed to the Cas sandwich protein.

In a third aspect of the assay method of the second aspect or any other aspect, wherein: the gRNA is an amine-conjugated gRNA; and the gRNA is attached to the immobilization surface, the immobilization surface comprising a carboxylated surface with EDC-NHS chemistry.

In a fourth aspect of the assay method of the second aspect or any other aspect, wherein: the gRNA comprises a biotin-labeled gRNA; and the immobilization surface comprises a streptavidin-coated surface.

In a fifth aspect of the assay method of the second aspect or any other aspect, wherein the Cas enzyme is complexed to the gRNA to form a non-cleaving complex.

In a sixth aspect of the assay method of the second aspect or any other aspect, wherein the plurality of secondary gRNAs are fluorescently labeled.

In a seventh aspect of the assay method of the second aspect or any other aspect, wherein the plurality of secondary gRNAs is modified by addition of at least haptens, biotin, fluorescent labels, quantum dots, nanoparticles, enzymes, and enzyme substrates.

In an eighth aspect of the assay method of the second aspect or any other aspect, wherein the plurality of secondary gRNAs is modified by signal amplification agents comprising at least a covalently labeling product with biotin conjugation and a streptavidin labeled-quantum dot.

In a ninth aspect of the assay method of the second aspect or any other aspect, wherein the plurality of secondary gRNAs is modified by addition of signal amplification agents comprising at least a covalently bonded labeling product with Cy5 conjugation and a mouse anti-Cy5 antibody and anti-mouse quantum dot.

In a tenth aspect of the assay method of the second aspect or any other aspect, wherein the Cas sandwich protein is modified to contain a tag that may be detected.

In an eleventh aspect of the assay method of the tenth aspect or any other aspect, wherein the Cas sandwich protein comprises the tag comprising a fluorescent protein, a hapten, biotin, or combinations thereof.

In a twelfth aspect of the assay method of the second aspect or any other aspect, wherein the immobilization surface comprises at least one of: a microarray; a Luminex bead; a magnetic bead; a 384-well plate; and a 96-well plate.

In a thirteenth aspect of the assay method of the twelfth aspect or any other aspect, wherein: the immobilization surface comprises the microarray; and the microarray is printed onto a functionalized glass surface.

In a fourteenth aspect of the assay method of the twelfth aspect or any other aspect, wherein; the assay method may be configured to detect the target nucleic acid; and the target nucleic acid is isolated or derived from a pathogen.

In a fifteenth aspect of the assay method of the twelfth aspect or any other aspect, wherein: the assay method may be configured to detect the target nucleic acid; and the target nucleic acid is isolated or derived from a mosquito.

In a sixteenth aspect of the assay method of the twelfth aspect or any other aspect, wherein the target nucleic acid is isolated from a sample from a subject.

In a seventeenth aspect of the assay method of the sixteenth aspect or any other aspect, wherein the sample from the subject comprises at least one of blood, urine, bone marrow, serum, saliva, semen, cerebrospinal fluid, oral fluid, stool, sputum, and tissue.

In an eighteenth aspect, the present disclosure includes a system for detecting a target pathogen comprising: an immobilization surface, a gRNA attached to the immobilization surface, a Cas enzyme complexed to the gRNA, and a target nucleic acid derived from a pathogen.

In a nineteenth aspect of the system of the eighteenth aspect or any other aspect, wherein the immobilization surface comprises at least one of: a microarray; a Luminex bead; a magnetic bead; a 384-well plate; and a 96-well plate.

In a twentieth aspect of the system of the nineteenth aspect or any other aspect, wherein the target nucleic acid may be labeled.

In a twenty-first aspect of the system of the twentieth aspect or any other aspect, wherein the target nucleic acid is labeled with a Cas sandwich comprising: a randomized gRNA library complexed with a Cas sandwich protein; the randomized gRNA library comprising a plurality of secondary gRNAs; the secondary gRNAs comprising a tail complexed to the Cas sandwich protein and are labeled.

In a twenty-second aspect of the system of the twenty-first aspect or any other aspect, wherein the system is configured as a multiplex assay for detecting multiple target nucleotides.

In a twenty-third aspect of the system of the twenty-first aspect or any other aspect, wherein the plurality of the secondary gRNAs are fluorescently labeled.

In a twenty-fourth aspect of the system of the twenty-first aspect or any other aspect, wherein the plurality of secondary gRNAs is modified by addition of haptens, biotin, fluorescent labels, quantum dots, nanoparticles, enzymes, or enzyme substrates.

In a twenty-fifth aspect, the present disclosure includes a method of performing a multiplex assay comprising: immobilizing a gRNA to an immobilization surface; complexing a Cas enzyme to the gRNA; adding a target nucleic acid; labeling the target nucleic acid; detecting the target nucleic acid without amplification; determining one or more results based on the detected target nucleic acid via a reader; and wherein: the target nucleic acid is labeled with a Cas sandwich comprising: a randomized gRNA library complexed with a Cas sandwich protein; the randomized gRNA library comprising a plurality of secondary gRNAs; the secondary gRNAs comprising a tail complexed to a Cas sandwich protein and are labeled; and the randomized gRNA library comprises secondary gRNAs comprising a sequence at least 80% identical to SEQ. ID NO. 1, SEQ. ID NO. 2, or SEQ ID. NO. 3.

In a twenty-sixth aspect, the present disclosure includes a method comprising: producing a library of gRNAs comprising a sequence at least 80% identical to SEQ. ID NO. 1, SEQ. ID NO. 2, or SEQ ID. NO. 3.

In a twenty-seventh aspect, the present disclosure includes a method comprising: using a library of gRNAs for nucleotide detection comprising a sequence at least 80% identical to SEQ. ID NO. 1, SEQ. ID NO. 2, or SEQ ID. NO. 3.

In a twenty-eighth aspect, the present disclosure includes a gRNA library comprising a sequence at least 80% identical to SEQ. ID NO. 1, SEQ. ID NO. 2, or SEQ ID. NO. 3.

In a twenty-ninth aspect of the assay method of the first aspect or any other aspect, wherein the gRNA comprises a tailed gRNA; and the immobilization surface comprises a surface with a capture probe complementary to the tail.

In a thirtieth aspect, the present disclosure includes a method for preparing an assay for detection of one or more target nucleic acids, the method comprising: immobilizing gRNA to a surface; complexing a Cas enzyme to the immobilized gRNA to form a non-cleaving complex; adding a target nucleic acid; labeling the target nucleic acid; and directly reading the unamplified results via a reader compatible with a format of the assay.

In a thirty-first aspect of the method of the thirtieth aspect or any other aspect, wherein: the gRNA is an amine-conjugated gRNA; and the surface comprises a carboxylated surface with EDC-NHS chemistry.

In a thirty-second aspect of the method of the thirtieth aspect or any other aspect, wherein: the gRNA comprises a biotin-labeled gRNA; and the surface comprises a streptavidin-coated surface.

In a thirty-third aspect of the method of the thirtieth aspect or any other aspect, wherein the surface comprises at least one of a microarray; a Luminex bead; a magnetic bead; a 384-well plate; and a 96-well plate.

In a thirty-fourth aspect of the method of the thirty-third aspect or any other aspect, wherein the microarray is printed onto a functionalized glass surface.

In a thirty-fifth aspect of the method of the thirtieth aspect or any other aspect, wherein: the Cas enzyme comprises dead Cas.

In a thirty-sixth aspect of the method of the thirtieth aspect or any other aspect, wherein: the CAS enzyme comprises an enzymatically active Cas protein; and the gRNA comprises dead gRNA.

In a thirty-seventh aspect of the method of the twenty-fifth aspect or any other aspect, wherein the SEQ ID. NO 3 comprises a sequence of TTTV prior to a scaffold sequence.

In a thirty-eighth aspect of the method of the twenty-seventh aspect or any other aspect, wherein the SEQ ID. NO 3 comprises a sequence of TTTV prior to a scaffold sequence.

In a thirty-ninth aspect of the method of the twenty-eighth aspect or any other aspect, wherein the SEQ ID. NO 3 comprises a sequence of TTTV prior to a scaffold sequence.

In a fortieth aspect of the gRNA library of the thirtieth aspect or any other aspect, wherein the SEQ ID. NO 3 comprises a sequence of TTTV prior to a scaffold sequence.

### BRIEF DESCRIPTION OF THE DRAWINGS

The foregoing Summary and the following Detailed Description will be better understood when read in conjunction with the appended drawings. In the drawings:

FIG. 1 is a flow diagram showing an assay method in accordance with one or more embodiments of the present disclosure.

FIG. 2 is a schematic of assay chemistry labeling a target nucleic acid sequence with a detectable marker before it is captured by a CRISPR-associated (Cas) protein complex, according to one embodiment of the present disclosure.

FIG. 3 is a schematic of assay chemistry labeling a target nucleic acid sequence with a detectable marker after it is captured by a CRISPR-associated (Cas) protein complex, according to one embodiment of the present disclosure.

FIG. 4 is a schematic of a system for signal amplification used to detect target nucleic acid with a labeling dye conjugated with a fluorophore, according to one embodiment of the present disclosure.

FIG. 5 is a schematic of a system for signal amplification used to detect target nucleic acid with a labeling dye conjugated with biotin followed by a streptavidin-conjugated quantum dot.

FIG. 6 is a schematic of a system for signal amplification including the Cas “sandwich” approach, which illustrates a detection of target nucleic acid using one or more Cas proteins complexed with biotin-labeled randomized gRNA, bound to one or more streptavidin-labeled quantum dots, according to one embodiment of the present disclosure.

FIG. 7 is a schematic of a system for signal amplification using anti-target primary antibodies bound to one or more fluorophore-conjugated secondary antibodies, according to one embodiment of the present disclosure.

FIG. 8 is a schematic of a system for signal amplification using anti-target primary antibodies, according to one embodiment of the present disclosure.

FIG. 9 is schematic of a system for signal amplification using CRISPR-based nucleic acid detection including a magnetic bead, an immobilized guide RNA, a Cas Protein, and at least one target nucleic acid, according to one embodiment of the present disclosure.

FIGS. 10A and 10B include a schematic of a system for signal amplification using CRISPR-based nucleic acid detection including the immobilized guide RNA, Cas protein, and at least one target nucleic acid of FIG. 9, and further including components for secondary capture and labeling and detection of a fluorescent signal, according to one embodiment of the present disclosure.

FIG. 11 is a schematic of a system for signal amplification using CRISPR-based nucleic acid detection including the immobilized guide RNA and Cas protein of FIGS. 9-10, wherein the target nucleic acid is labeled with a primary antibody, anti-Cy3 via Mirus and a secondary antibody is bound to the primary antibody includes a labeled detection mechanism, according to one embodiment of the present disclosure.

FIG. 12A is a graph illustrating the relative fluorescence units (RFU) as a function of a concentration of a target nucleic acid specific to Chlamydophila pneumonia, detected by the system of FIG. 11, according to one embodiment.

FIG. 12B is a graph illustrating a signal relative fluorescence unit (RFU) as a function of concentration of a target nucleic acid specific to Influenza A, H1N1, according to one embodiment of the present disclosure.

FIG. 12C is a chart illustrating a limit of detection for various assays and their target type.

FIGS. 13A-13D illustrate signal RFU values indicating inclusivity and exclusivity values for a DNA virus (e.g., orthopox) variants of the SARS-CoV-2 virus, various antimicrobial resistance (AMR) genes, and the H1N1 influenza A virus.

FIG. 14 is a schematic of an exemplary system using CRISPR-based nucleic acid detection for detecting a nucleotide target labeled with fluorescent dye, according to one embodiment.

FIGS. 15A-15D illustrate the normalized fluorescence units (RFU) associated with detection of various nucleotide sequences detected by the exemplary system of FIG. 14, according to one embodiment.

FIG. 16 is a schematic of an exemplary system using CRISPR-based nucleic acid detection for detecting a nucleotide target labeled with a Cas sandwich protein, according to one embodiment.

FIG. 17 illustrates the dose dependent mean fluorescence intensity (MFI) associated with detection of various amounts of total copies of C. pneumoniae detected by the system of FIG. 16, according to one embodiment.

FIG. 18 is a schematic of an exemplary system using CRISPR-based nucleic acid detection for detecting a synthetic nucleotide target labeled with a fluorescent label on each end of the nucleotide target, according to one embodiment

FIG. 19 depicts identification of the presence of C. pneumoniae and B. holmesii using a microarray assay designed using the system of FIG. 16, according to one embodiment.

FIG. 20 is a schematic of an exemplary system using CRISPR-based nucleic acid detection comprising at least a magnetic bead and designed for detecting a nucleotide target which is biotinylated, according to one embodiment.

FIGS. 21A-21D illustrate the dose dependent values of normalized median MFI associated with various amounts of total copies of C. pneumoniae, B. holmesii, and antimicrobial resistance markers detected by the system of FIG. 20, according to one embodiment.

FIGS. 22A-22C illustrate relative fluorescence units (RFU) of associated with detection of orthopox and PhiX detected by an embodiment of the system, according to one embodiment.

FIG. 23 illustrates variables to be considered in a method of designing a secondary gRNA library, according to one embodiment.

FIG. 24 is an oligonucleotide table that identifies sequences of oligonucleotides used in secondary gRNA library design, according to one embodiment.

### DETAILED DESCRIPTION

In various embodiments, CRISPR-based detection assays (e.g. DETECTR™ and SHERLOCK™) utilize a live Cas protein/gRNA complex that, upon target activation, utilizes trans-activation of the enzyme to nonspecifically cleave nearby reporter molecules, generating a signal. Since the reporter is nonspecific, the CRISPR assay may not be easily multiplexed to detect a large number of targets within a single reaction. Furthermore, the assay relies upon enzyme turnover to generate a signal, so assay sensitivity will increase the longer the reaction is allowed to proceed (until it comes to completion). Therefore, the sensitivity is limited by time. This is mitigated by pre-amplifying the target sequence prior to detection by the Cas-complex. Therefore, the CRISPR assay uses a pre-amplification step to increase target signal to improve sensitivity- and CRISPR detection via the Cas protein/gRNA complex for specificity. This adds complexity to assay design and limits some applications of the technology. The current assays function essentially a reporter system for amplicon detection (analogous to TaqMan or Molecular Beacons) rather than a true and reliably sensitive target detection method.

CRISPR technologies have revolutionized the field of molecular biology by providing a precise and efficient method for genetic modification, enabling the development of highly specific and sensitive PCR assays. By leveraging CRISPR's ability to target and modify specific DNA sequences, researchers can enhance the accuracy and reliability of PCR assays for various applications, including pathogen detection, genetic diagnostics, and research on genetic variations. Pathogen outbreaks including the Ebolavirus, SARS-CoV-2 and Mpox virus outbreaks have shown, novel, emerging, and re-emerging pathogens present an ever-evolving challenge that require a rapid development of PCR panels. It is desirable to utilize CRISPR technologies to quickly reconfigure panels to accommodate new pathogens and/or new sequence variations within 24 hours of discovery.

As used herein, the terms immobilize and immobilization refer to the process or act of attaching or fixing a molecule, typically a nucleic acid (e.g., DNA or RNA) or a protein, to a solid support or surface (this is in contrast to molecules being in solution). In various embodiments, immobilization is important in Cas (CRISPR-associated) assays to capture and detect the target molecule of interest. In Cas assays, the target molecule (e.g., DNA or RNA) is may be immobilized onto a solid surface, including a microarray or a well of a microplate. In various embodiments, the surface may include at least one of a microarray, a Luminex bead, a magnetic bead, a 96-well plate, and a 384-well plate. This immobilization may allow for efficient and specific capture of the target molecule, which may then be detected and analyzed using the CRISPR-associated components, including Cas proteins or guide RNA. Immobilization may refer to various different methods, including but not limited to physical adsorption, covalent binding, or affinity-based interactions, and other suitable methods of immobilization. Immobilization may help facilitate the interaction between the target molecule and the Cas assay components, enabling sensitive and specific detection of the molecule of interest.

As used herein, a Cas complex (also Cas protein/gRNA complex) can refer to the combination of a Cas protein with a guide RNA (gRNA) molecule. This complex is able to bind to target nucleic acid based on complementarity of the gRNA to the target nucleic acid. Upon binding to target nucleic acid, the Cas complex can cleave one or both strands of the target nucleic acid.

As used herein, dead Cas may refer to a Cas protein that is catalytically inactive. A Cas complex that uses a dead Cas protein will be able to bind target nucleic acids specifically but will not be able to cleave the target. Cas protein, Cas enzyme, Cas, and CRISPR-associated endonuclease are used herein interchangeably.

As used herein, a guide RNA (gRNA), may refer to an RNA molecule that contains two regions: a scaffold region that is used to complex the gRNA with the appropriate type of Cas protein, and a guide region that is complementary to a target nucleic acid. The length and sequence of the scaffold and guide regions are dictated by the specific types of Cas proteins. A gRNA library is a collection of gRNAs that allow binding to multiple target nucleic acids.

As used herein, dead gRNA may refer to a gRNA having specific alterations in its sequences resulting in a target/gRNA mismatch, such that a complex of Cas enzyme and dead gRNA will bind to the target but will fail to trigger cleavage by the Cas enzyme. Dead gRNA, dgRNA, and d-gRNA are herein used interchangeably, but are distinct from references to gRNA without the qualifier “dead.”

As used herein, target nucleic acid may refer to DNA or RNA molecules of interest for detection. The target sequence contains a short protospacer that is complementary to the guide region. Adjacent to the protospacer is the protospacer adjacent motif (PAM) sequence, which is crucial for target recognition and cleavage. The PAM sequence, typically a short 2-6 base pair sequence, is recognized by the Cas protein. The nature of the PAM sequence varies with the type of Cas protein; for instance, Cas9 generally recognizes a ‘NGG’ PAM, Cas12 recognizes a ‘TTTV’ PAM, and Cas13 does not require a PAM. The PAM is only required for guiding the Cas complex to the target sequences and is not found in the gRNA guide region.

As used herein, Cas12, Cas12, previously known as Cpf1, is a Cas protein. Distinguished from other proteins like Cas9, Cas12 recognizes a ‘TTTV’ Protospacer Adjacent Motif (PAM) sequence, located upstream of the target DNA. Additionally, Cas12 creates staggered cuts in DNA, leaving ‘sticky ends’, which can simplify the insertion of new DNA sequences. Another distinguishing feature is its ability to function using a single crRNA, obviating the need for an additional tracrRNA. Uniquely, Cas12 also possesses the ability to target and cleave single-stranded DNA, and, post activation, remains active to degrade nearby ssDNA.

As used herein, Cas13, (previously known as C2c2) is a specific type of Cas protein. Unlike other Cas proteins including Cas9 and Cas12 that target DNA, Cas13 specifically targets RNA, thereby offering a distinct method for gene manipulation without altering the DNA sequence. Cas13 recognizes specific RNA sequences with the help of a guide RNA, cleaving the target RNA upon recognition. Furthermore, activated Cas13 displays collateral activity, where it indiscriminately cleaves nearby non-target RNAs. Cas13 also does not require a PAM.

Various embodiments of the disclosure utilize a “dead” version of the Cas protein/gRNA complex that will bind to specific target nucleic acids based on gRNA design, but not cleave the target. To allow multiplexing, Cas protein/gRNA complexes may be immobilized to an assay detection surface, with each spot representing a specific Cas/gRNA combination. After adding target nucleic acids to the detection area, target nucleic acids may be captured on the surface by a specific Cas protein/gRNA complex within a location. Target visualization can occur by detecting binding events at the capture site. This may be achieved by one of several methods that fluorescently label target sequences prior to or after capture by Cas protein/gRNA complexes. Therefore, the chemistry of the systems and methods described herein is designed so the assay is immobilized and detected at the same location. Furthermore, the chemistry of the systems and methods described herein utilizes “direct” detection; where direct detection is described as detection where there is no pre-amplification of target material prior to detection via the Cas protein/gRNA complex.

The following distinctions of the disclosed systems and methods may enable multiple advantages over prior assays:

Massively Multiplexed Assays. Various aspects of the disclosed systems and methods enable very high multiplexing (at least hundreds of assays).

Immobilization and detection on one spot. The technology of the systems and methods can be applied to an array-based format, where hundreds of assays (or potentially more) can be multiplexed together. Similarly, the systems and methods may be amenable to configuration with Luminex technology (or similar suitable technologies), where assays designed against different targets are immobilized onto different beads (with each bead type being equivalent to an array spot) that are individually distinguishable by the instrument and multiplexed together.

Direct detection (no pre-amplification) is a major advantage of the methods and systems described herein. In some embodiments, the systems and methods are easy to multiplex, as there is no need to design primers against all of the various targets. In some cases, there can be a limitation to how many primers that can multiplex together without cross-reactivity, which is why PCR and other amplification-based methods are not highly multiplexed.

Rapid Time to Result. The systems and methods as described herein may be configured to function with a very rapid time to result, since the system and methods only rely upon target binding, and not enzyme turnover, for signal. Therefore, in some embodiments of the systems and methods, the time to result is only dependent upon how long it takes for the target to be labeled and detected.

Rapid Assay Reconfigurability. The systems and methods as described herein may be highly suited to be easily reconfigurable within a very short time period. In some embodiments, the direct detection of the target is key to this aspect, as may enable in silico evaluation of assays with better prediction of performance, without the need to empirically test the gRNA's compatibility with primer pairs. Furthermore, in some embodiments, the systems and methods may can utilize machine learning to facilitate assay design and predictivity.

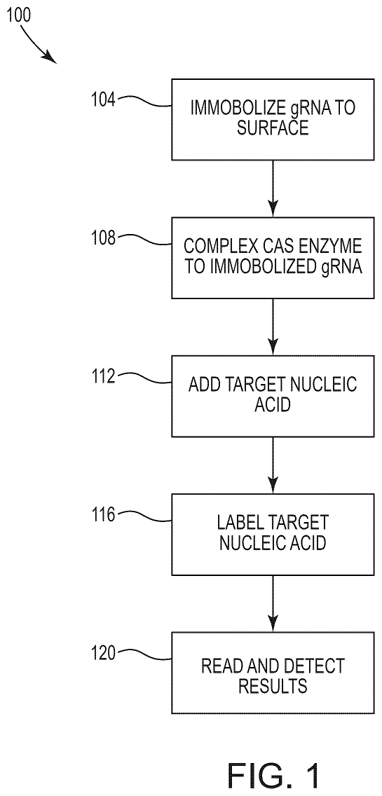

FIG. 1 is a flow diagram showing a method of preparing and reading an assay 100 in accordance with one or more embodiments of the present disclosure. The method 100 includes a step 104 of immobilizing at least one of a gRNA to a surface. In various embodiments, the step 104 may immobilize at least two gRNAs to a surface. In some embodiments, amine-conjugated gRNA may be used and covalently immobilized to a carboxylated surface with EDC-NHS chemistry. Examples of suitable carboxylate surfaces include but are not limited to a microarray including microarrays printed onto a functionalized glass surface, a bead including Luminex® or magnetic beads, a plate including a 384 or 96 well plate, or any other suitable surface. In some embodiments biotin-labeled gRNA may be used and immobilized to a streptavidin-coated surface. In some embodiments, chemical immobilization of the gRNA may be achieved by other interactions, including but not limited to streptavidin-biotin, epoxide-amine, thiol-gold, hydrazide, aldehyde, maleimide, or the like.

The method 100 includes a step 108 of complexing the Cas enzyme to the immobilized gRNA to form a non-cleaving complex. In some embodiments, dead Cas proteins may be used to create a gRNA/Cas complex that can bind to a target specific to the gRNA, but which would not result in cleavage upon binding. In some embodiments, an enzymatically active Cas protein may be used in conjunction with a dead gRNA such that the target/gRNA mismatch allows for binding to the target without activation of the Cas protein. In some embodiments, the amino acid sequence of a Cas protein may be altered such that in can bind to specific target nucleic acid sequences without cleaving. In some embodiments, a Cas protein may be used under reaction conditions or with components that allow binding of target sequences without cleaving. In some embodiments, Cas proteins of various types may be one of the following types: Cas9, Cas12, Cas13, or Cas14. In some embodiments, various subtypes of Cas proteins may be used; for example, known subtypes of Cas12 include Cas12a, Cas12b, Cas12c, Cas12d, Cas12e, Cas12f, Cas12g, Cas12h, Cas12i, Cas12j (with discovery of new subtypes, this list will continue to grow). In some embodiments, catalytically dead sequence-specific nucleases may be as Zinc Finger Nucleases (ZFNs) and Transcription Activator-Like Effector Nucleases (TALENs)

The method 100 includes a step 112 of adding the target nucleic acid. In some embodiments, the target nucleic acid may comprise extracted total nucleic acid. Such extracted total nucleic acid may be extracted manually via an extraction kit or as part of an automated sample preparation (ASP) device or an ASP module within another device, including a Massively Multiplexed Assay Device (MMD). In various embodiments, other target nucleic acids may be used, including but not limited to extracted DNA, extracted total RNA, mRNA, non-coding RNAs, or other analytes that contain a nucleic acid tag recognizable by a Cas protein/gRNA complex.

The method 100 includes a step 116, of labeling the target nucleic acid. In various embodiments, any suitable labeling method or labeling agent may be used. In some embodiments, labeling is provided via a Cas sandwich.

As used herein, a Cas sandwich refers a target nucleic acid labeling method. The Cas sandwich approach provides detection via a randomized gRNA library complexed with at least one Cas sandwich protein. In various embodiments, the randomized gRNA library complexed with at least two Cas sandwich proteins. In some embodiments, target nucleic acids may be captured by immobilized Cas complexes. In some embodiments, immobilized target sequences are bound to a solution phase library of Cas complexes containing a randomized library of gRNAs. In some embodiments, the randomized gRNAs are fluorescently labeled. In other embodiments, the randomized gRNA library is designed so that any captured target nucleic acid may have protospacers complementary to some members of the gRNA library. In effect, the captured target may be sandwiched between the immobilized Cas complex and the solution phase Cas/random gRNA complexes. In additional embodiments, the fluorescent label on the randomized gRNAs allows fluorescent signal to be associated with a specific capture spot. In some embodiments, gRNAs are labeled with a hapten including biotin. In some embodiments, streptavidin-labeled quantum dots bind to the biotin-labeled gRNA to produce signal amplification without target amplification.

As used herein, randomized gRNA library refers to a gRNA library that may, in various embodiments, be fully randomized, be partially randomized, contain universal bases including inosine, may consist of pool of gRNA sequences enriched for sequences expected to be contained in the target nucleic acid.

Such a gRNA library may be labeled with many alternative modifications, including but not limited to at 5′ end, internal, or at 3′ end. Modifications may be one of many haptens (e.g., biotin), fluorescent labels, quantum dots, nanoparticles, enzymes, enzyme substrates. Multiple modifications may be incorporated into one gRNA.

Various combinations of fluorescent probe and signal amplification agents may be used including but not limited to, a covalently labeling product such Mirus dye with biotin conjugation, followed by a streptavidin labeled-quantum dot can bind to the biotin for signal amplification or Mirus dye with Cy5 conjugation, followed by mouse anti-Cy5 antibody and anti-mouse quantum dot.

Various other nucleic acid dye beyond those discussed above may be used, including but not limited to Qubit, YoYo®, Ribogreen®, and the like or combinations thereof. Methods using such dyes may provide faster signal results, which may be valuable good for certain applications, it may provide less sensitive, resulting in a higher limit of detection.

One having ordinary skill in the art will recognize that different Cas proteins may be relied upon in different contexts. By way of example but not limitation, Cas 13 may be utilized in the context of RNA, whereas Cas 12 may be utilized in the context DNA.

In some embodiments, the Cas protein may be modified to contain a tag that may be detected directly or indirectly. Some tags may include but not be limited to a fluorescent protein (including green or red fluorescent protein or other suitable fluorescent proteins), haptens, biotin, and other tags such as 6×HIS, and the like or combinations thereof.

In some embodiments, the Cas protein may be modified to contain functional domains comprising RNA binding domains or DNA binding domains that may increase the efficiency of target capture or binding.

The method 100 includes a step 120 of reading the results by detecting a signal in one or more assays. In some embodiments, the signal may be luminescence, fluorescence, or any other suitable signal that may be detected. In some embodiments, results may be read directly without amplification due to the labeling method. In some embodiments, assay design may enable reading of the results at the same region of the immobilization surface to which the gRNA was initially immobilized. In this way, results associated with different targets may be distinguishable based on their region on the surface. In some embodiments, the results may be read using any suitable reader based on the signal. One having ordinary skill in the art will recognize that any reader compatible with the assay format (i.e. immobilization surface) may be used. Examples of possible readers include but are not limited to microarray readers for microarray slides, an MMD reader for assays incorporated into an MMD cartridge, a Luminex reader for Luminex beads including Luminex 200, MAGPIX, Intelliflex and the like, a fluorimeter for magnetic beads or plate-based assay, and any other suitable readers.

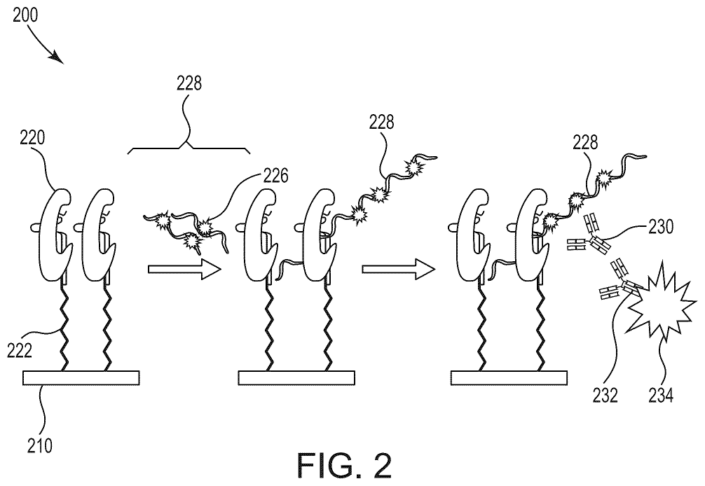

FIG. 2 is a schematic of a system 200 for labeling a target nucleic acid sequence with a detectable marker before it is captured by a CRISPR-associated (Cas) protein complex, according to one embodiment of the present disclosure.

In general, FIG. 2 illustrates labeling of one or more target nucleotide sequences before the Cas complex is introduced for capture, ensuring that only labeled target sequences are captured by the Cas complex during the subsequent capture step. This approach allows for specific capture of the labeled target sequences, potentially reducing background noise and improving the efficiency of the capture process.

The system 200 includes a one or more Cas proteins 220 complexed to a gRNA 222 that is immobilized against the immobilized surface 210. In various embodiments, and as shown in FIG. 2, the one or more Cas proteins 220 are “dead” Cas proteins in a Cas protein/gRNA complex immobilized against the immobilized surface 210.

The system may include one or more target nucleotide sequences 228 conjugated with a labeling dye 226. The one or more target nucleotide sequences 228 conjugated with a labeling dye 226 can be introduced to the system where the Cas protein/gRNA complex is active, where the Cas9/gRNA complex binds to the labeled target nucleotide sequence(s) 228 through the complementarity between the gRNA and the labeled target nucleotide sequence(s) 228. Further, the interaction between the labeled target nucleotide sequence(s) 228 and the Cas protein/gRNA complex can be visualized using various detection techniques. In various embodiments, and as illustrated and described in FIG. 2, the interaction can be visualized using fluorescence microscopy. In various embodiments, the labeled target nucleotide sequence(s) 228 can be introduced to the cas9/gRNA complex in vitro (in a test tube) or in vivo (within one or more cells).

Furthermore, the system 200 may include a primary antibody 230 for recognizing the labeling dye 226, the target nucleotide sequence 228, and/or the Cas protein 220. In various embodiments, the primary antibody 230 may be bound to the labeling dye 226 by adding the primary antibody 230 to the cas9-gRNA complex and washing any unbound primary antibody from the solution. In various embodiments, the primary antibody includes at least one of anti-Cy3, anti-ss-DNA, and anti-ssRNA.

Further yet, the system 200 may include a secondary antibody 232 that is conjugated to a fluorophore 234. In various embodiments, the secondary antibody 232 is specific to the primary antibody 230. Furthermore, in various embodiments, the secondary antibody 232 that is conjugated to the fluorophore can be added to the primary antibody 230 in solution with any unbound secondary antibody 232 washed out in a washing step. Signal detection can be performed with the final system including the Cas protein/gRNA complex, the target nucleotide sequence 228, the labeling dye 226, the primary antibody 230, the secondary antibody 232, and the fluorophore 234 conjugated to the secondary antibody 232 using methods including fluorescence microscopy, which utilizes the secondary antibody 232 conjugated to the fluorophore 234, wherein the fluorophore 234 is excited by light when the sample is illuminated by a light. The light emitted through the excitation of the fluorophore 234 can be captured with a camera or fluorescence microscope and the fluorescence intensity can be analyzed using image analysis software to quantify the presence of the target nucleotide sequence 228. In various instances, a fluorometer may be used for detection of fluorescence. In various embodiments, alternative methods to detect and/or visualize the labeled biomolecules, may include at least one of colorimetric detection, chemiluminescence, electrochemical detection, surface plasmon resonance, mass spectrometry, ELISA, and qPCR.

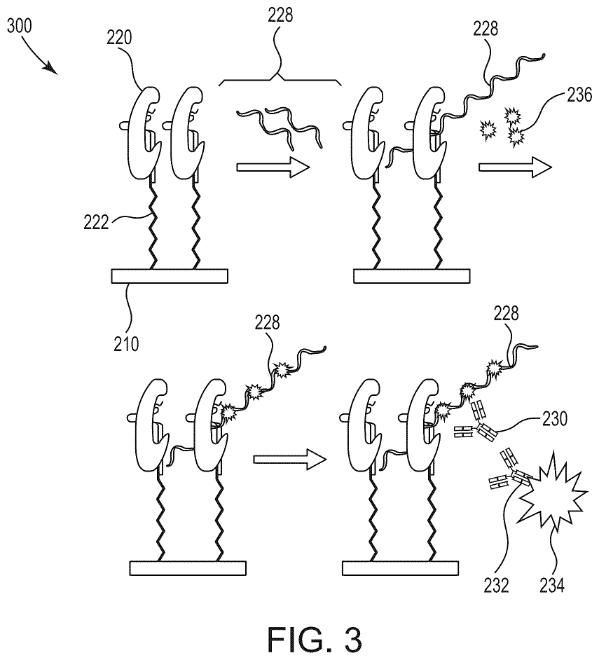

FIG. 3 is a schematic of a system 300 for labeling a target nucleic acid sequence 228 with a detectable marker (e.g., a labeling dye 226) after it is captured by a CRISPR-associated (Cas) protein complex, according to one embodiment of the present disclosure.

In general, FIG. 3 illustrates a method 300 of post-capture labeling including attaching a labeling dye 226 to the captured target nucleotide sequences 228 after the capture step has been completed. FIG. 3 includes many of the components illustrated and described above with respect to FIG. 2. In various embodiments, the target nucleotide sequences 228 are first captured by the Cas protein/gRNA complex and then the labeling dye 226 or antibodies 230, 232 are introduced to bind to the captured target nucleotide sequences 228. This described method may offer flexibility in labeling, as it allows for the labeling of a wide range of captured targets. Additionally, the method may allow for the labeling of multiple targets simultaneously.

In various embodiments, pre-capture labeling may ensure that only the intended target sequences are labeled and subsequently captured. In contrast, post-capture labeling may capture non-target sequences along with the intended targets, requiring additional specificity controls. Furthermore, pre-capture labeling may offer higher capture efficiency for the labeled targets compared to post-capture labeling, as it directly incorporates the label into the target sequences before capture. In some embodiments, the design of a method using pre-capture and/or post-capture labeling may depend on the specific experimental requirements, including the desired sensitivity, specificity, and workflow considerations.

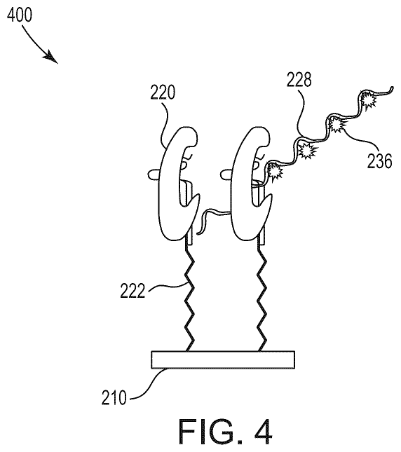

FIG. 4 is a schematic of a system 400 for signal amplification used to detect target nucleic acid 228 with a labeling dye 226 conjugated with a fluorophore 234, according to one embodiment of the present disclosure.

As also illustrated above with respect to FIGS. 2-3, the system 400 may include a Cas protein/gRNA complex having a Cas protein 220 and a gRNA 222 immobilized to an immobilized surface 210. In contrast to the system 200, 300 of FIGS. 2-3, in some embodiments, the Cas protein/gRNA complex may be directed to a target nucleic acid 228 that includes a labeling dye 226 conjugated with a fluorophore 234 (i.e., the system 400 does not include a secondary antibody 232). In various embodiments, the labeling dye 226 can include one or more of Cy3, Cy5, rhodamine, fluorescein, or other labeling dyes as recognizable by one of ordinary skill in the art.

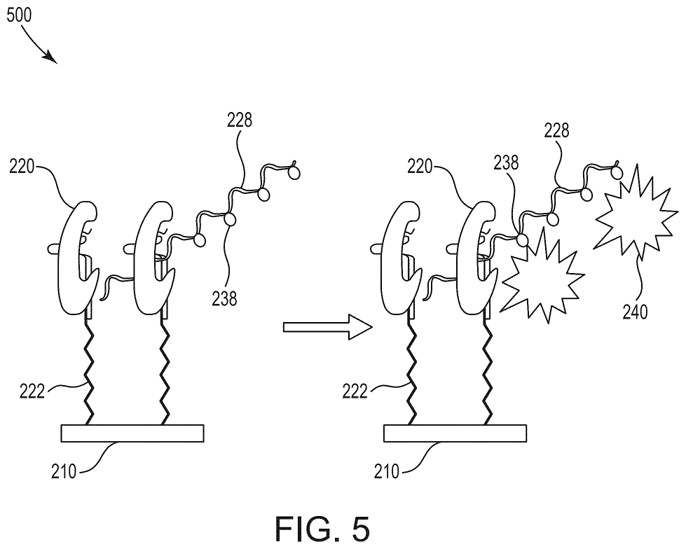

FIG. 5 is a schematic of a system 500 for signal amplification which can be used to detect target nucleic acid 228 with a labeling dye conjugated with biotin 238 followed by a streptavidin-conjugated quantum dot 240.

The system 500 includes similar components as illustrated and described above with respect to FIGS. 2-4, including the Cas protein 220, the gRNA 222 (which forms the Cas protein/gRNA complex), the immobilized surface 110, the target nucleic acid 228, and the labeling dye 226. Further, the system 500 includes a labeling dye conjugated with biotin 238 and a streptavidin-conjugated quantum dot 240 bound to the labeling dye conjugated with biotin 238.

In various embodiments, the labeling dye 226 is conjugated with biotin by mixing the dye with biotin molecules in a reaction buffer containing one or more appropriate reagents. Under specific conditions (including a range of pH and temperature), the reactive groups on the labeling dye 226 and biotin molecules can form stable covalent bonds that result in the conjugation of the labeling dye 226 with biotin. The conjugated product (the labeling dye conjugated with biotin 238) can be purified to remove any unreacted dye or biotin molecules in solution to ensure purity of the final conjugate.

In various embodiments, streptavidin, a protein that binds specifically to biotin with high affinity, can be conjugated with quantum dots, which are semiconductor nanocrystals used to emit fluorescent light when excited by an external light source. In various embodiments, the quantum dots are used as highly sensitive fluorescent labels to indicate the binding of the streptavidin to the target labeling dye conjugated with biotin 238. The purpose of combining the streptavidin-conjugated quantum dots 240 with the labeling dye conjugated with biotin 238 can be for multiplexed imaging or detection applications. In some embodiments, by using streptavidin-biotin interactions, multiple types of labeled biomolecules (each conjugated with biotin) can be simultaneously visualized using quantum dot labels. In various embodiments, the simultaneous visualization may allow for the detection of multiple targets within a single sample, providing valuable information about complex biological systems, including protein-protein interactions, gene expression patterns, or cellular localization studies, with high sensitivity and specificity.

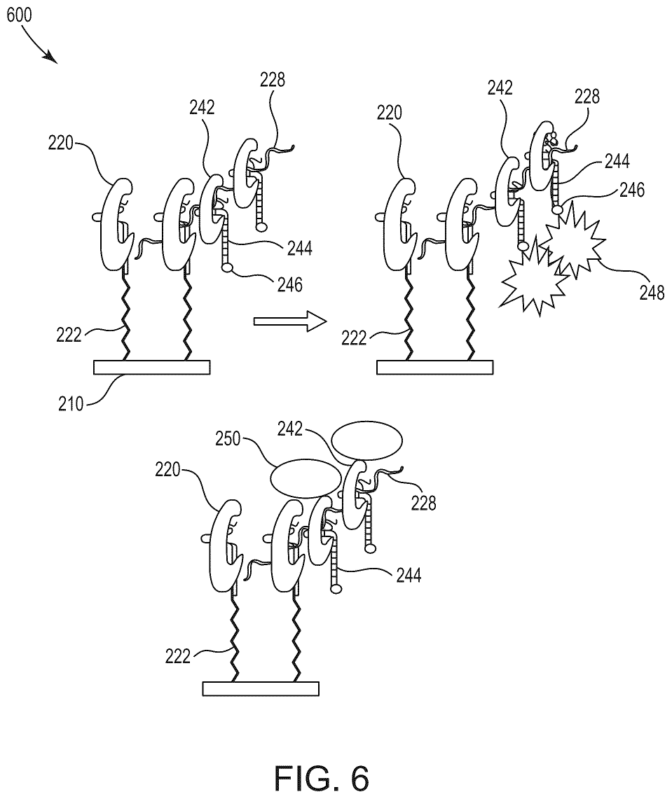

FIG. 6 is a schematic of a system 600 for signal amplification including the Cas “sandwich” approach, which illustrates a detection of target nucleic acid using one or more Cas proteins complexed with biotin-labeled randomized gRNA, bound to one or more streptavidin-labeled quantum dots, according to one embodiment of the present disclosure.

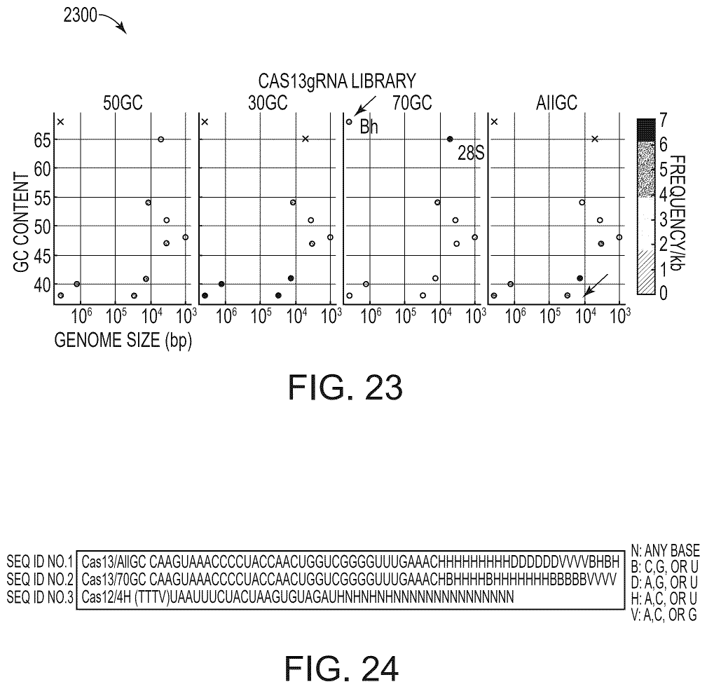

The system 600 may include at least one Cas sandwich protein 242. In some embodiments, the system 600 includes at least two Cas sandwich proteins 242. In additional embodiments, the system may include are multiple Cas proteins 220 on either side of the target nucleotide sequence 228. In various embodiments, the Cas sandwich proteins 242 may be complexed with one or more random secondary guide RNAs 244 from a guide RNA library, which is illustrated and described below in further detail with respect to FIGS. 23-24. In various embodiments, the randomized gRNA can include a biotin label 246 or fluorophores 234.

Furthermore, in various the system 600 can include the addition of a complex having a streptavidin-conjugated quantum dot 248 conjugated to the biotin label 246 for the purpose of signal amplification.

Further yet, the system 600 can include the addition of a complex including a GFP-conjugated Cas sandwich, which may be used to visualize and track the localization and movement of the Cas protein within cells or biological samples. In some embodiments, by fusing GFP 250, a fluorescent protein, to the Cas protein, researchers can monitor the dynamics of Cas protein behavior, including its subcellular localization, interaction with target nucleotide sequences, and kinetics of genome editing processes like CRISPR-Cas9-mediated DNA cleavage and repair. In various embodiments, the indication of subcellular localization may enable real-time monitoring and visualization of Cas protein interactions with DNA or RNA targets, aiding in various applications including gene editing, nucleic acid detection, and studying cellular processes.

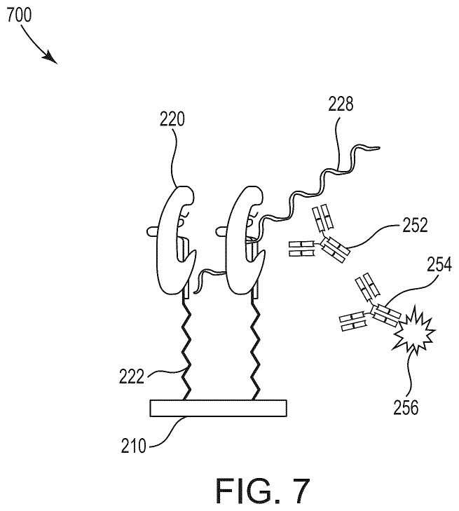

FIG. 7 is a schematic of a system 700 for signal amplification using anti-target antibodies 252 bound to one or more secondary antibodies 254 conjugated to a fluorophore 256, according to one embodiment of the present disclosure. In various embodiments, the anti-target antibodies 252 can target single or double stranded RNA. In various embodiments, the anti-target antibodies 254 can include an antibody for targeting ssRNA or dsDNA.

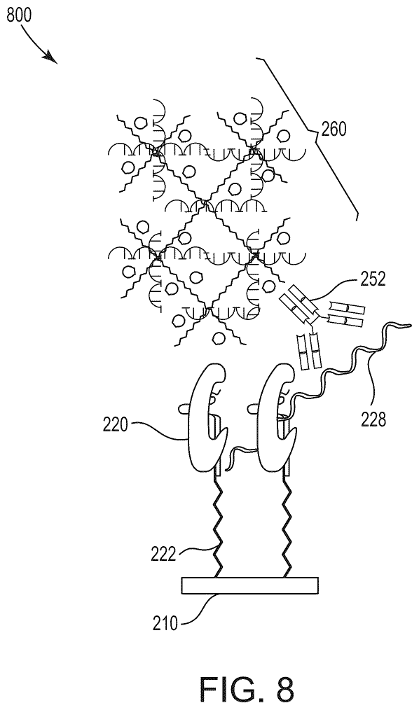

FIG. 8 is a schematic of a system 800 for signal amplification using anti-target antibodies 252 followed by aptamer/nanoparticle 260 signal amplification, according to one embodiment of the present disclosure. Aptamer and nanoparticle amplification may involve utilizing aptamers, which are short single-stranded DNA or RNA molecules that bind specifically to target molecules, as recognition elements. In various embodiments, aptamers may be conjugated to nanoparticles, including for example gold or magnetic nanoparticles, to enhance signal detection. In some embodiments, when the aptamer-nanoparticle complex binds to the target molecule, it may cause signal amplification through the accumulation of nanoparticles, which may enable sensitive detection of the target molecule in various assays, including diagnostics and biosensing.

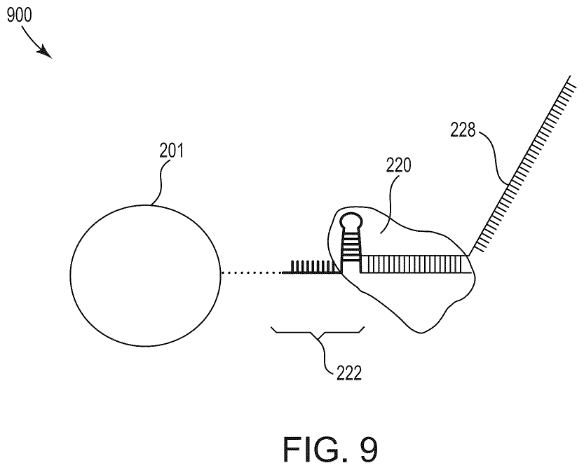

FIG. 9 is schematic of a system for signal amplification using CRISPR-based nucleic acid detection including a magnetic bead, an immobilized guide RNA, a CAS Protein, and at least one target nucleic acid, according to one embodiment of the present disclosure. In various examples, the Cas protein 220 is a “dead” Cas protein as illustrated and described above.

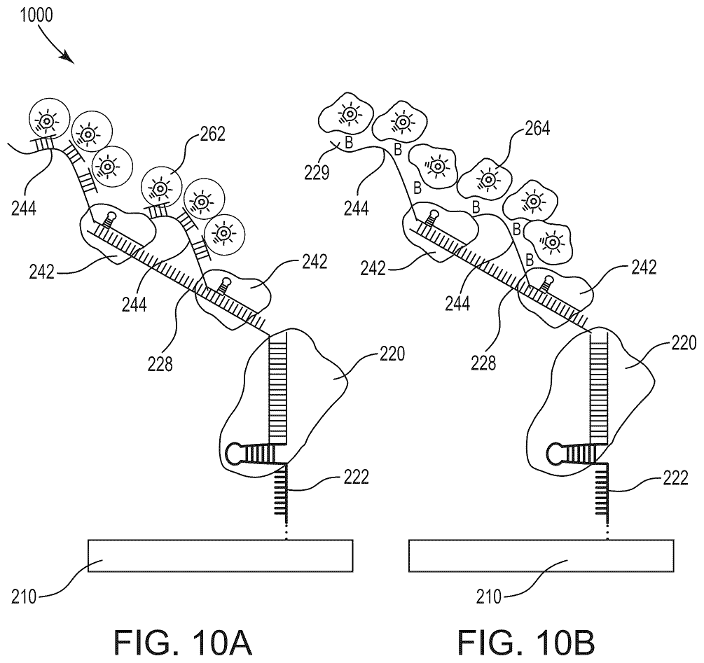

FIG. 10 is a schematic of a system 1000 for signal amplification using CRISPR-based nucleic acid detection including the immobilized guide RNA, Cas protein, and at least one target nucleic acid of FIG. 9, and further including components for secondary capture and labeling and detection of a fluorescent signal, according to one embodiment of the present disclosure.

In general, FIG. 10 shows the addition of a tail to the secondary gRNA to aid in detection. In some embodiments, adding a tail to a secondary gRNA can serve to aid in detection. In various embodiments, the tail sequence may be unique to different secondary gRNAs. In some embodiments, the tail sequence may be designed to identify each gRNA during detection, allowing for multiplexed assays where multiple gRNAs can be detected simultaneously. In various embodiments, the secondary gRNA is a tailed gRNA and the immobilization surface 210 comprises a surface with a capture probe complementary to the tail.

The system 1000 includes many of the components as illustrated and described above with reference to FIG. 7, including the immobilized guide RNA 222, the “dead” Cas protein 220, and the target nucleic acid 228. Furthermore, the system 1000 may include a hybridized probe with a label 262 on the tail to aid in detection as described above.

The system 1000 of FIG. 10B may include similar components to the system 1000 of FIG. 10A, including the immobilized guide RNA 222, the “dead” Cas protein 220, and the target nucleic acid 228. Further, the system 1000 of FIG. 10B may include a secondary biotinylated gRNA with a tail complexed to the dead Cas protein 220. Further yet, the system 1000 of FIG. 10B may include a streptavidin-phycoerythrin or other anti-biotin detection molecule for detecting the biotinylated gRNA. In various embodiments, the tail of the system 1000 is configured to utilize Mirus to add biotin or fluorophores to the tail.

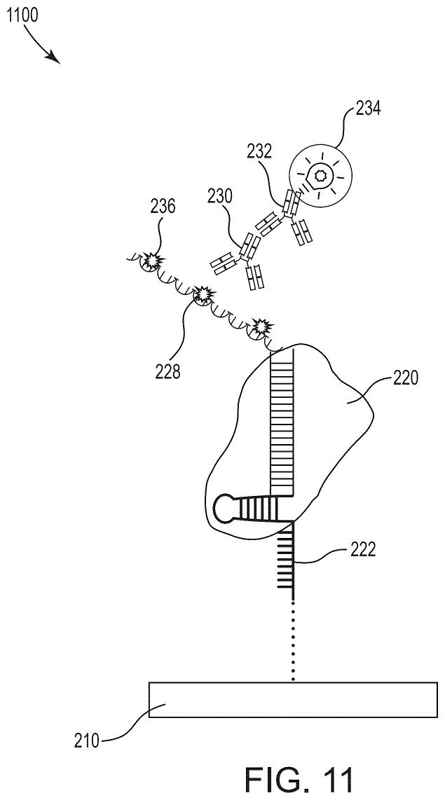

FIG. 11 is a schematic of one embodiment of a system 1100 for detecting a nucleotide target. The system 1100 for detecting a nucleotide target may have the same types of system components as the detection system 200 of FIG. 4 (wherein similar components have like reference numbers) but may have fewer system components and/or system components in a different configuration than the detection system 200 of FIG. 2. In particular, the detection system 1100 may include the immobilized surface 210, the gRNA 222, the Cas protein 220, the target nucleotide sequence 228, and the labeling dye 226. Furthermore, the system 1100 may include a fluorescent labeling dye 236, a primary antibody, a secondary antibody, and a fluorophore 234 conjugated to the secondary antibody 232.

In some embodiments, the system 1100 may be designed as an immobilized plate-based assay or a bead-based assay. In some instances, the immobilized plate-based assay or a bead-based assay comprises the surface 210. In various embodiments, the surface 210 may comprise at least one of a microarray, a Luminex bead, a magnetic bead, a 384-well plate, and a 96-well plate. In some instances of the detection system 1100, if formatted as a microarray, the microarray may be printed onto a functionalized glass surface.

In some embodiments, the gRNA 222 may be immobilized and attached to the surface 210. In various embodiments, the gRNA 222 may include an amine-conjugated gRNA attached to the surface 210 comprising a carboxylated surface with EDC-NHS chemistry.

In some embodiments, the target nucleotide sequence 228 may be labeled with a labeling dye 228. In various embodiments, the labeling dye 226 may be applied to substantially label the target nucleotide sequence 228. In some embodiments, the labeling dye 266 is a fluorescent dye. In further embodiments, the labeling dye 266 may comprise a Qubit® dye. In further embodiments of the detection method 1400, the detection of the target nucleotide sequence 228 may be measured using a Qubit® fluorometer or any other suitable fluorometer or reader.

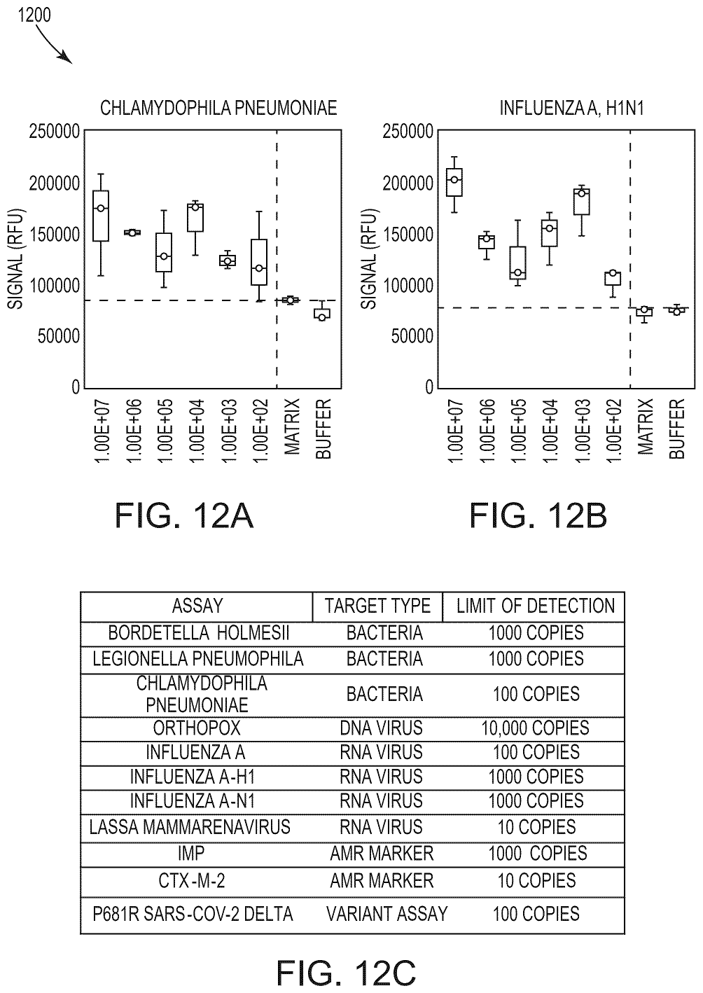

FIGS. 12A-12C provide graphs 1200 of fluorescence intensity. The detection system 1200 may be used to detect the presence of any nucleotide sequence. The system 1200 may be used to detect nucleotide sequences derived from any pathogen or human. The system 1200 may be used to detect nucleotide sequences derived from any pathogen indicating microbial resistance, for example the genes IMP, CTX-M-2. In some embodiments, the detection system 1100 may be used to detect Bordetella holmesii, Leionella pneumophila, Chlamydophila pneumoniae, Orthopox, Influenza A, Influenza A-H1, Influenza A-N1, Lassa fever (e.g., Mammarenavirus), IMP, CTX-M-2, any variant of Covid-19, and P681R SARS CoV-2 Delta. These target assays can include a target type (e.g., bacteria, DNA virus, RNA virus, antimicrobial resistance (AMR) marker, or variant assay) and a limit of detection (in copies) as illustrated.

FIGS. 12A and 12B illustrate data collected from the system 1100 of FIG. 11. In FIGS. 12A and 12B, the signal of detection (in RFU) is shown relative to a concentration of Chlamydophila pneumonia and Influenza A, H1N1. As can be seen in the graphs, the targets illustrate a dose-dependent response, meaning the signal intensity substantially increases as the concentration of labeled assay targets is increased. Further, a negative control (e.g., the buffer and matrix solutions) exhibits low fluorescence intensity relative to the labeled assays.

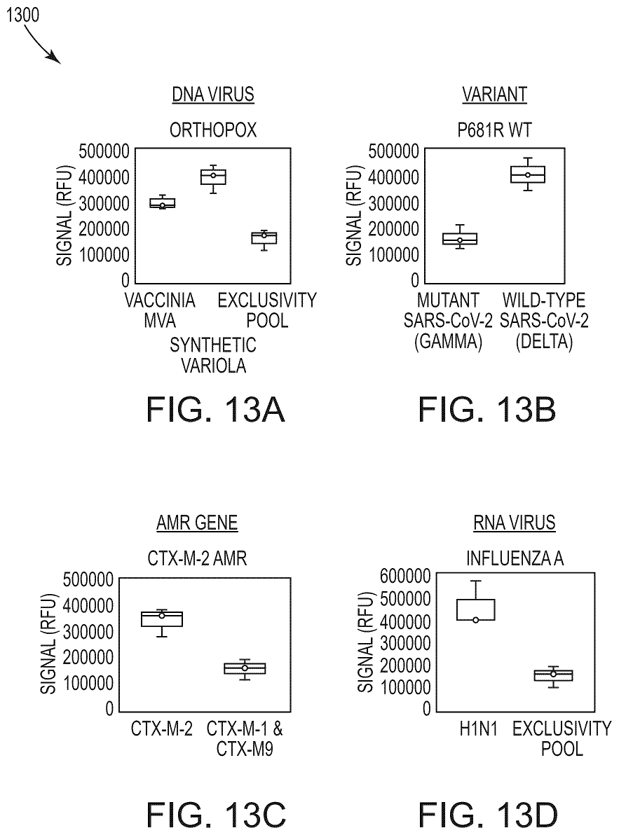

FIGS. 13A-13D illustrates data collected from the system 1100 of FIG. 11. FIG. 13 illustrates inclusivity and exclusivity data for a DNA virus (e.g., orthopox) variants of the SARS-CoV-2 virus, various antimicrobial resistance (AMR) genes, and the H1N1 influenza A virus. In some embodiments, evaluating inclusivity may ensure that a diagnostic test or detection method accurately identifies all relevant target sequences or organisms, maximizing sensitivity and reducing false negatives. Evaluating exclusivity ensures the test does not cross-react with non-target sequences or organisms, thereby minimizing false positives and enhancing specificity. In FIG. 13, relatively high inclusivity is demonstrated by showing the targeted variants (e.g., Orthopox and Influenza A) exhibit much greater luminescence than the exclusivity pool. Furthermore, relatively high exclusivity is demonstrated by showing the targeted variants (e.g., Synthetic Variola, Wild-Type Sars-CoV-2, and CTX-M-2) exhibit greater luminescence compared to non-target sequences of similar variants, including for example Vaccinia MVA, mutant Sars-CoV-2, and CTX-M-1 & CTX-M-9.

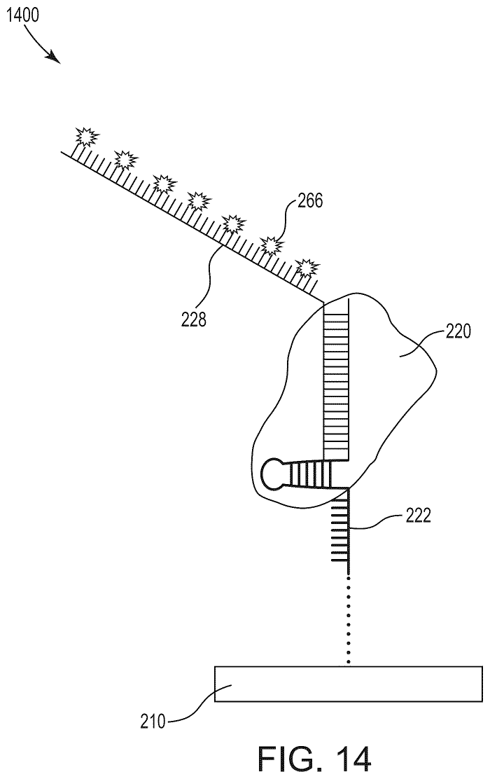

Turning to FIG. 14, one embodiment of a system 1400 for detecting a nucleotide target sequence herein is provided. The system 1400 for detecting a nucleotide target may have the same types of system components as the detection system 200 of FIG. 2 (wherein similar components have like reference numbers) but may have fewer system components and/or system components in a different configuration than the detection system 200 of FIG. 2. In particular, the detection system 1400 may include the immobilized surface 210, the gRNA 222, the Cas protein 220, the target nucleotide sequence 228, and the labeling dye 226.

In some embodiments, the system 1400 may be designed as an immobilized plate-based assay or a bead-based assay. In some instances, the immobilized plate-based assay or a bead-based assay comprises the surface 210. In various embodiments, the surface 210 may comprise at least one of a microarray, a Luminex bead, a magnetic bead, a 384-well plate, and a 96-well plate or any other suitable format. In some instances of the detection system 1400, if formatted as a microarray, the microarray may be printed onto a functionalized glass surface.

In some embodiments, the gRNA 222 may be immobilized and attached to the surface 210. In various embodiments, the gRNA 222 may be an amine-conjugated gRNA attached to the surface 210 comprising a carboxylated surface with EDC-NHS chemistry.

In another embodiment, the Cas protein 220 may be a dead Cas protein.

In some embodiments, the target nucleotide sequence 228 may be labeled with a labeling dye 228. In various embodiments, the labeling dye 226 may be applied to substantially label the target nucleotide sequence 228. In some embodiments, the labeling dye 266 is any suitable fluorescent dye. In further embodiments, the labeling dye 266 may comprise a Qubit® dye. In further embodiments of the detection method 1400, the detection of the target nucleotide sequence 228 may be measured using a Qubit® fluorometer or any other suitable fluorometer or reader.

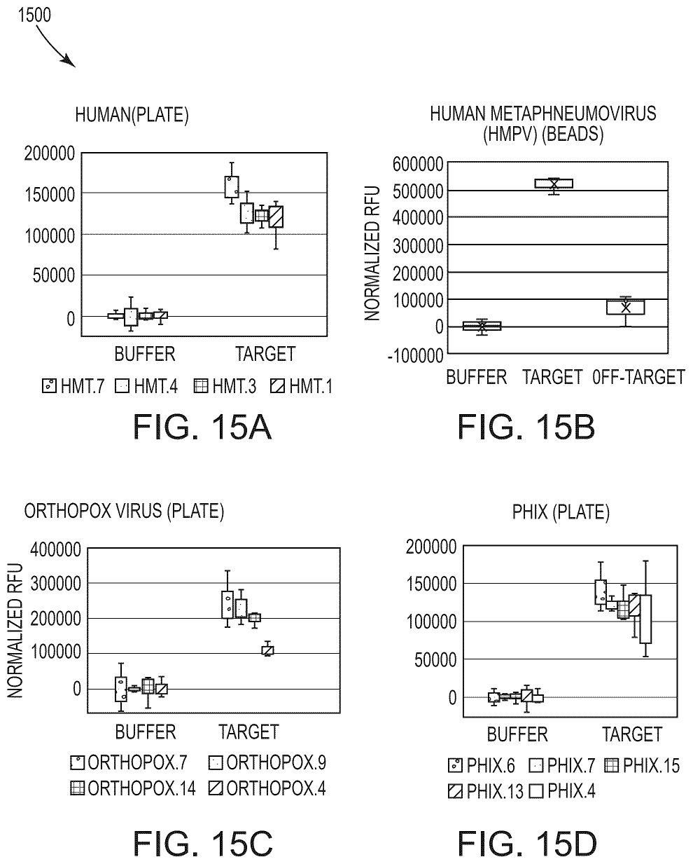

Turning now to FIGS. 15A-15D which provide graphs 1500 of fluorescence intensity, the detection system 1400 may be used to detect the presence of any nucleotide sequence. In some embodiments, the detection system 1400 may be used to detect human sequences, human metapneumovirus (HMPV), orthopox virus, and PhiX via the observation of an increase in normalized RFU compared to the control (i.e. the buffer).

In FIG. 15A, the detection system 1400 may be designed as a plate-based assay to detect an increased RFU in the presence of human nucleotide sequences corresponding to h28.7, hMt.4, hMt.3, and hMt.1. In FIG. 15B, the detection system 1400 may be designed as a bead-based assay to detect an increased normalized RFU in the presence of target HMPV nucleotides. In some instances, the detection system 1400 is sensitive enough to significantly identify target nucleotide sequences compared to off target sequences, which do not produce a substantially increased amount of RFU compared to the control. In FIG. 15C, the detection system 1400 may be designed as a plate-based assay to detect an increased normalized RFU in the presence of target orthopox virus nucleotides. In FIG. 15D, the detection system 1400 may be designed as a plate-based assay to detect an increased normalized RFU in the presence of PhiX nucleotides.

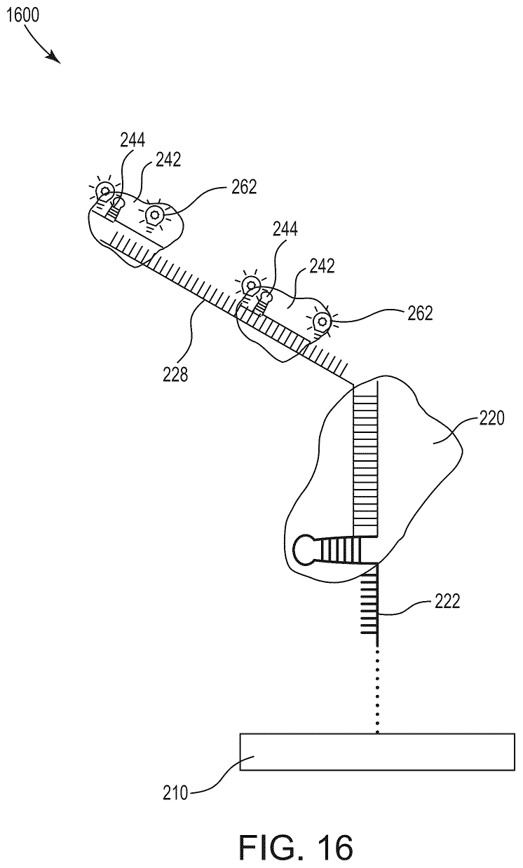

Referring to FIG. 16, one embodiment of a system 1600 for detecting a nucleotide target sequence herein is provided. The system 1600 for detecting a nucleotide target may have the same types of system components as the detection system 200 of FIG. 2 and the detection system 600 of FIG. 6 (wherein similar components have like reference numbers) but may have fewer system components and/or system components in a different configuration than the detection system 200 and 600. In particular, the detection system 1600 may include the immobilized surface 210, the gRNA 222, the Cas protein 220, the target nucleotide sequence 228, the Cas sandwich protein 242, the secondary gRNAs, and the hybridized probe with a label 262.

In some embodiments, the system 1600 may be designed as an immobilized plate-based assay or a bead-based assay. In some instances, the immobilized plate-based assay or a bead-based assay comprises the surface 210. In various embodiments, the surface 210 may comprise at least one of a microarray, a Luminex bead, a magnetic bead, a 384-well plate, and a 96-well plate. In some instances of the detection system 1600, if formatted as a microarray, the microarray may be printed onto a functionalized glass surface. In various instances, the detection system 1600 may be designed as a bead based Luminex® assay. In yet another instance, the system 1600 may be designed to use a printed microarray slide.

In some embodiments, the gRNA 222 may be immobilized and attached to the surface 210. In various embodiments, the gRNA 222 may include an amine-conjugated gRNA attached to the surface 210 comprising a carboxylated surface with EDC-NHS chemistry.

In another embodiment, the Cas protein 220 may comprise a dead Cas protein and be bound to the immobilized gRNA 222. In various embodiments, the target nucleotide sequence 228 may be detected using at least one Cas sandwich protein 242. In other embodiments the target nucleotide sequence 228 is detected using at least two Cas sandwich proteins 242 in a complex with at least one secondary gRNA 244 from a randomized gRNA library. In some embodiments, the secondary gRNA 244 comprises a tail complexed with the Cas sandwich proteins 242. In additional embodiments, the secondary gRNA 244 comprises the hybridized probe 262 with a label.

In some embodiments, the hybridized probe 262 may comprise any suitable hybridized probe 262. In other instances, the hybridized probe 262 may include In further embodiments of the detection method 1600, the detection of the target nucleotide sequence 228 may be measured using any suitable reader including a fluorometer.

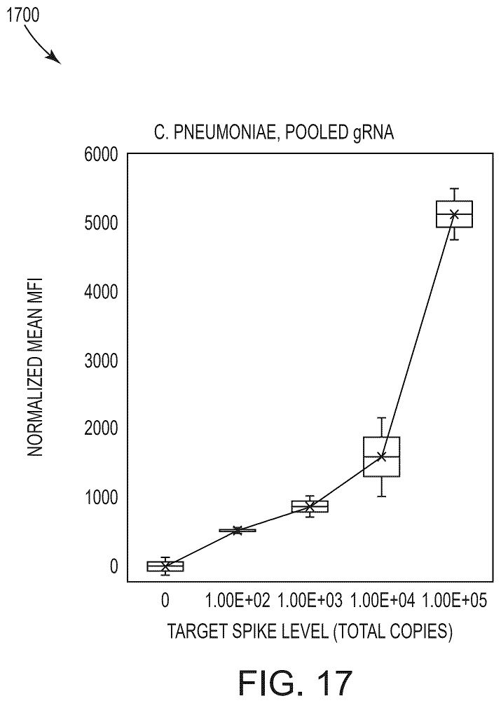

Turning to FIG. 17, graph 1700 shows that the detection system 1600 may be designed as a bead-based assay to detect an increased normalized mean MFI upon presence of C. pneumoniae. In various embodiments of the detection system 1600, the system 1600 can be designed as a Luminex® bead-based test wherein pooled gRNAs 222 may be immobilized to Luminex® beads and complexed to the dead Cas protein 220. In some instances, different copy amounts of C. pneumoniae can be added to demonstrate a dose dependent detection whereby the more target nucleotide sequence 228 is present, the higher the detected normalized mean MFI. In some embodiments, the detection system 1600 may be designed to include a randomized library of secondary gRNAs 244 wherein the secondary gRNAs 244 can be labeled with ATTO-550, complexed to the dead Cas 220, immobilized on a Luminex® based assay, and read with a Luminex® MAGPIX system.

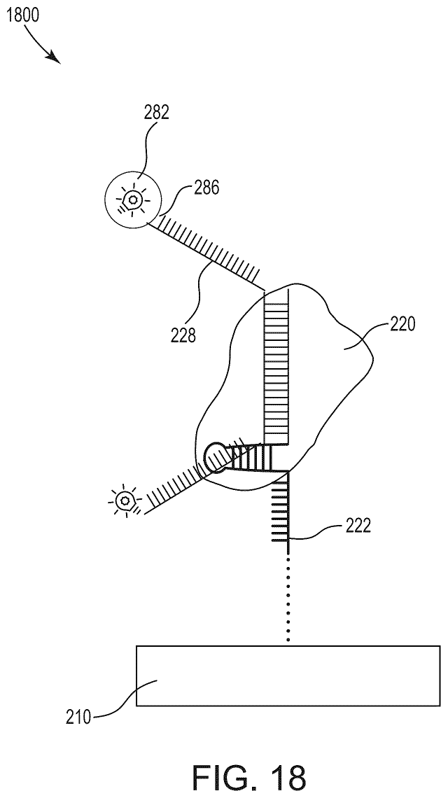

Referring now to FIG. 18, one embodiment of a system 1800 for detecting a nucleotide target sequence herein is provided. The system 1800 for detecting a nucleotide target may have the same types of system components as the detection system 200 of FIG. 2 (wherein similar components have like reference numbers) but may have fewer system components and/or system components in a different configuration than the detection system 200. In particular, the detection system 1800 may include the immobilized surface 210, the gRNA 222, the Cas protein 220, the target nucleotide sequence 228, and at least two fluorescent probes.

In some instances, the detection system 1800 may include the target nucleotide sequence 228 wherein the target nucleotide sequence 228 is synthetic. In some instances, the synthetic target nucleotide sequence 228 comprises a first end 286 and a second end 288. In additional embodiments, the first end 286 is labeled with a first fluorescent label 282. In another embodiment, the second end 288 is labeled with a second fluorescent label 284. The first and second fluorescent labels 282, 284 may include any suitable fluorescent label.

In various embodiments, the detection system 1800 may be designed to be performed and analyzed using a Massively Multiplexed Detection (MMD) device.

In some embodiments, the detection system 1800 includes at least the gRNA 222 printed and/or immobilized in an array format and assembled into a cartridge, the dead Cas protein 220, and the fluorescently labeled synthetic nucleotide target 228.

In some embodiments, the detection system 1800 may include at least the gRNA 222 printed and/or immobilized in an array format and assembled into a cartridge. In additional instances, dead Cas protein 220 may be added to the cartridge, followed by the addition of a fluorescently labeled synthetic nucleotide target 228.

Turning to FIG. 19, in some instances, the cartridge can be read and analyzed by the MMD device. In various embodiments, the detection system 1800 can be configured as a multiplex, in some instances configures as a 400-plex detection system. In additional embodiments, synthetic C. pneumoniae could be added to the cartridge and detected in a multiplex format. In some instances, biotin may be used as a control.

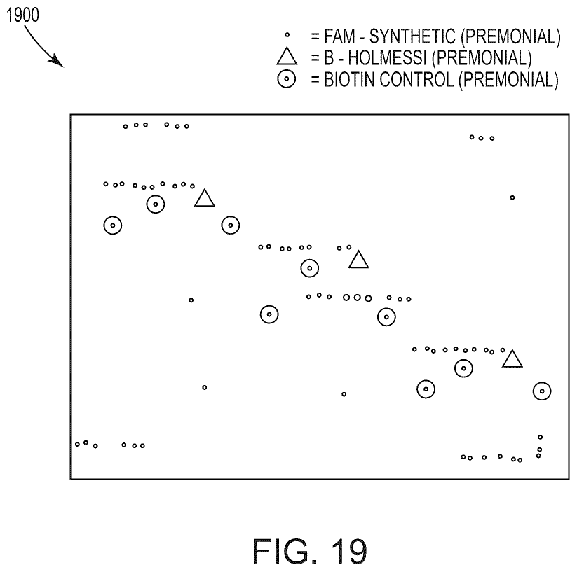

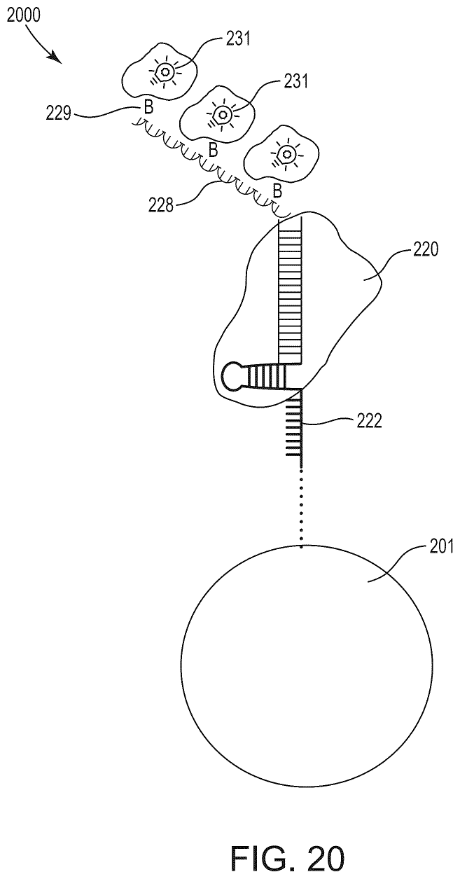

Turning to FIG. 20, one embodiment of a system 2000 for detecting a nucleotide target sequence herein is provided. The system 2000 for detecting a nucleotide target may have the same types of system components as the detection system 200 of FIG. 2 and detection system 900 of FIG. 9 (wherein similar components have like reference numbers) but may have fewer system components and/or system components in a different configuration than the detection system 200, 900. In particular, the detection system 2000 may include the magnetic bead 201, the immobilized surface 210, the gRNA 222, the Cas protein 220, the target nucleotide sequence 228, a biotinylated region 229 of the target nucleic acid 228, and at least one streptavidin-phycoerythrin (SAPE) component.

In various embodiments, the target nucleotide sequence 228 comprises a biotinylated region 229 wherein the target nucleotide sequence 228 may be biotinylated using mirus Bio® Label-IT. In various embodiments, the Cas protein 220 is a dead Cas protein 220. In additional embodiments, the target nucleotide sequence 228 is detected upon addition of the SAPE component which may allow for binding to the biotinylated region 229 of the target nucleotide sequence 228. In additional embodiments, the detection system 2000 may be designed to be configured and read using a Luminex® instrument or any other suitable instrument.

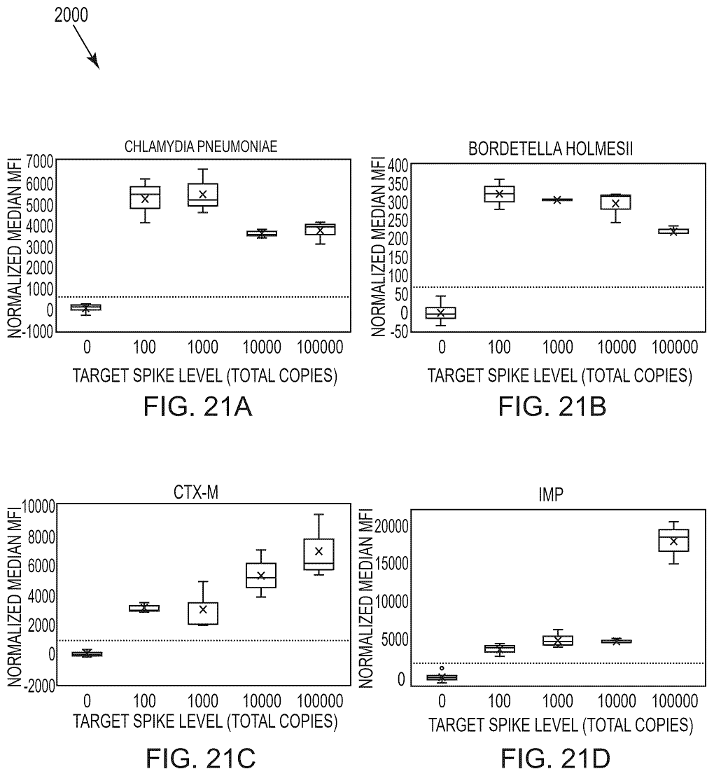

Referring now to FIG. 21, which provide graphs 2100 of fluorescence intensity, the detection system 2000 may be used to detect the presence of any nucleotide sequence. In some embodiments, the detection system 2000 may be used to detect C. pneumoniae, B. holmesii, and nucleotide sequences indicative of antimicrobial resistance (e.g., CTX-M and IMP) via the observation of an increase in normalized median MFI compared to the control (0 copies).

In FIG. 21A, the detection system 2000 may detect an increased normalized median MFI upon the presence of a target spike level of C. pneumoniae nucleotide sequences. The dotted line indicates 2× the standard deviation of the no target spike. In FIG. 121B, the detection system 2000 may be designed to detect an increased normalized median MFI upon the presence of a target spike level of B. holmesii nucleotide sequences. In some instances, the detection system 2000 is sensitive enough to significantly identify target nucleotide sequences compared to off target sequences. In FIG. 21C and FIG. 21D, the detection system 2100 may be designed to detect an increased normalized median MFI upon the presence of the target spike level of nucleotide sequences for the CTX-M and IMP genes, which are known antimicrobial resistance markers. In various embodiments, the systems and methods of the disclosure as described herein may be used to detect at least one antimicrobial resistance markers. In yet another embodiment, the systems and methods of the disclosure as described herein may be used to detect more than one antimicrobial resistance markers.

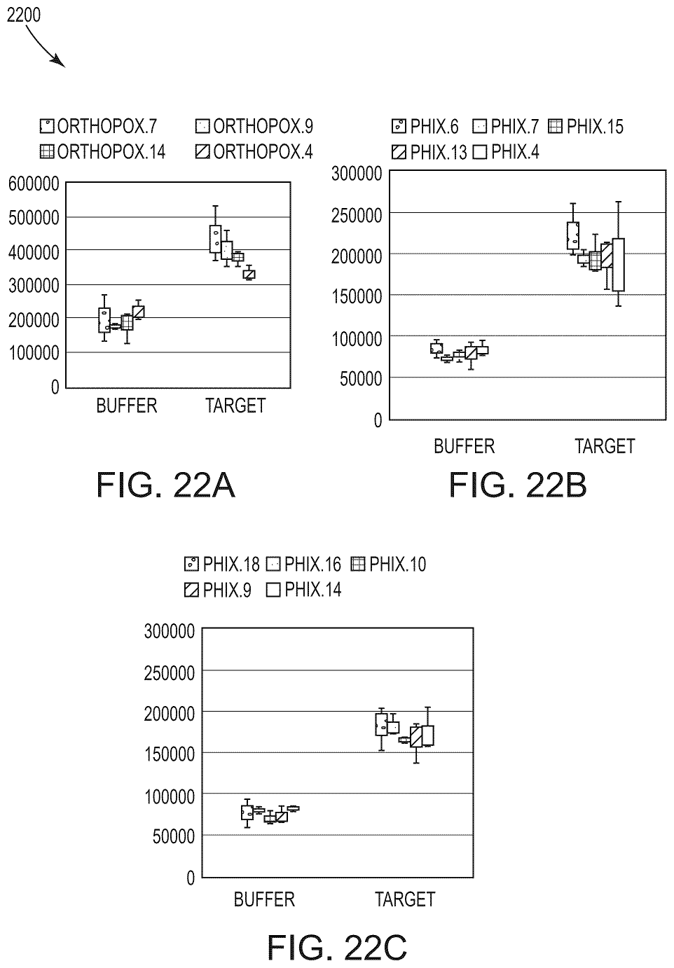

Turning to FIGS. 22A-22C, which provide graphs 2100 of fluorescence intensity, the methods and systems as described in this disclosure may be used to detect the presence of any nucleotide sequence. In some embodiments, the methods and systems of the disclosure may be used to detect the presence of orthopox and PhiX via the observation of an increase in RFU compared to the control (i.e., buffer).

Referring now to FIG. 23, which provides graph 2300 illustrating the frequency of gRNA presence (frequency/kb) as determined by the relationship between the GC content of the secondary gRNA library and the genome size (bp) of the target organism or pathogen (for systems including Cas 13). In yet another embodiment, the frequency of secondary gRNA 244 binding to the target nucleotide sequence 228 is dependent on GC content and genome size.

In various embodiments of the method and systems of the disclosure, the randomized gRNA library comprises a plurality of secondary gRNAs 244. In some embodiments, the randomized gRNA library is designed wherein the GC content of the gRNA is at about 30%, about 40%, about 50%, about 60%, and about 70%. In additional embodiments, the randomized gRNA library is designed wherein the GC content of the gRNA is at least 30%, at least 40%, at least 50%, at least 60%, and at least 70%.

Turning to FIG. 24, in various embodiments, the system comprises a randomized gRNA library comprising a library of gRNAs designed using the template sequence comprising the sequence of at least SEQ. ID 1, SEQ. ID 2, and SEQ ID. 3.

In one embodiment, a method of designing a randomized gRNA library comprises designing a library of gRNAs using the template sequence comprising the sequence of at least SEQ. ID 1, SEQ. ID 2, and SEQ ID. 3.

In various embodiments, the method of designing a randomized gRNA library comprises designing a library of gRNAs using a template sequence comprising a sequence that is at least 50% identical to SEQ. ID 1, SEQ. ID 2, or SEQ ID. 3.

In various embodiments, the method of designing a randomized gRNA library comprises designing a library of gRNAs using a template sequence comprising a sequence that is at least 60% identical to SEQ. ID 1, SEQ. ID 2, or SEQ ID. 3.

In various embodiments, the method of designing a randomized gRNA library comprises designing a library of gRNAs using a template sequence comprising a sequence that is at least 70% identical to SEQ. ID 1, SEQ. ID 2, or SEQ ID. 3.

In various embodiments, the method of designing a randomized gRNA library comprises designing a library of gRNAs using a template sequence comprising a sequence that is at least 80% identical to SEQ. ID 1, SEQ. ID 2, or SEQ ID. 3.

In various embodiments, the method of designing a randomized gRNA library comprises designing a library of gRNAs using a template sequence comprising a sequence that is at least 90% identical to SEQ. ID 1, SEQ. ID 2, or SEQ ID. 3.

In various embodiments, the method of designing a randomized gRNA library comprises designing a library of gRNAs using a template sequence comprising a sequence that is at least 95% identical to SEQ. ID 1, SEQ. ID 2, or SEQ ID. 3.

In many instances, SEQ. ID 1, SEQ. ID 2, and SEQ ID. 3 comprise a scaffold region comprising a fixed nucleotide sequence and a randomized guide sequence comprising a degenerate nucleotide sequence. Degenerate nucleotide sequence may describe a sequence wherein in one nucleotide position, multiple nucleotides could be chosen (e.g., N could indicate any base and B could indicate C, G, or U). FIG. 24 identifies that in the degenerate nucleotide sequence, N indicates that any base may be used; B indicates that C, G, or U may be used; D indicates that A, G, or U may be used; H indicates that A, C, or U may be used; and V indicates that A, C, or G may be used.

In multiple embodiments, SEQ. ID 1 may be used for systems comprising a Cas protein 220 including Cas13. In other embodiments, SEQ. ID 2 may be used for systems comprising a Cas protein 220 including Cas13. In additional embodiments, SEQ. ID 3 may be used for systems comprising a Cas protein 220 including Cas12.

In yet another embodiment, the systems may include a randomized secondary gRNAs 244 in a randomized gRNA library wherein the guide sequence does not interfere with scaffold folding, does not have excessive folding, and the random sequence of the gRNA sequences should be sufficiently degenerate to be present multiple times per captured target sequence.

### CONCLUSION

The foregoing description of the exemplary embodiments has been presented only for the purposes of illustration and description is not intended to be exhaustive or to limit the compositions, systems, and methods herein to the precise forms disclosed. Many modifications and variations are possible considering the above teachings.

The embodiments were chosen and described in order to explain the principles of the technology discussed herein and their practical application to enable others skilled in the art to utilize the various embodiments and with various modifications as are suited to the particular use contemplated. Alternative embodiments will become apparent to those skilled in the art to which the present technologies pertain without departing from their spirit and scope.

What is claimed is:

1. An assay method for detection of target nucleic acid, the method comprising:
immobilizing a guide RNA (gRNA) to an immobilization surface;
complexing a Cas enzyme to the gRNA;
adding a target nucleic acid;
labeling the target nucleic acid;
detecting the target nucleic acid without amplification; and
determining one or more results based on the detected target nucleic acid via a reader.

2. The method of claim 1, wherein:
the target nucleic acid is labeled with a Cas sandwich protein comprising a randomized gRNA library complexed with the Cas sandwich protein;
the randomized gRNA library comprises a plurality of secondary gRNAs; and
the plurality of secondary gRNAs are labeled and comprise a tail complexed to the Cas sandwich protein.

3. The method of claim 2, wherein:
the gRNA is an amine-conjugated gRNA; and
the gRNA is attached to the immobilization surface, the immobilization surface comprising a carboxylated surface with EDC-NHS chemistry.

4. The method of claim 2, wherein:
the gRNA comprises a biotin-labeled gRNA; and
the immobilization surface comprises a streptavidin-coated surface.

5. The method of claim 2, wherein the Cas enzyme is complexed to the gRNA to form a non-cleaving complex.

6. The method of claim 2, wherein the plurality of secondary gRNAs are fluorescently labeled.

7. The method of claim 2, wherein the plurality of secondary gRNAs is modified by addition of at least haptens, biotin, fluorescent labels, quantum dots, nanoparticles, enzymes, and enzyme substrates.

8. The method of claim 2, wherein the plurality of secondary gRNAs is modified by signal amplification agents comprising at least a covalently labeling product with biotin conjugation and a streptavidin labeled-quantum dot.

9. The method of claim 2, wherein the plurality of secondary gRNAs is modified by addition of signal amplification agents comprising at least a covalently bonded labeling product with Cy5 conjugation and a mouse anti-Cy5 antibody and anti-mouse quantum dot.

10. The method of claim 2, wherein the Cas sandwich protein is modified to contain a tag that may be detected.

11. The method of claim 10, wherein the Cas sandwich protein comprises the tag comprising a fluorescent protein, a hapten, biotin, or combinations thereof.

12. The method of claim 2, wherein the immobilization surface comprises at least one of:
a microarray;
a Luminex bead;
a magnetic bead;
a 384-well plate; and
a 96-well plate.

13. The method of claim 12, wherein:
the immobilization surface comprises the microarray; and
the microarray is printed onto a functionalized glass surface.

14. The method of claim 12, wherein;
the assay method may be configured to detect the target nucleic acid; and
the target nucleic acid is isolated or derived from a pathogen.

15. The method of claim 12, wherein:
the assay method may be configured to detect the target nucleic acid; and
the target nucleic acid is isolated or derived from a mosquito.

16. The method of claim 12, wherein the target nucleic acid is isolated from a sample from a subject.

17. The method of claim 16, wherein the sample from the subject comprises at least one of blood, urine, bone marrow, serum, saliva, semen, cerebrospinal fluid, oral fluid, stool, sputum, and tissue.

18. A system for detecting a target pathogen comprising:
an immobilization surface,
a gRNA attached to the immobilization surface,
a Cas enzyme complexed to the gRNA, and
a target nucleic acid derived from a pathogen.

19. The system of claim 18, wherein the immobilization surface comprises at least one of:
a microarray;
a Luminex bead;
a magnetic bead;
a 384-well plate; and
a 96-well plate.

20. The system of claim 19, wherein the target nucleic acid may be labeled.

21. The system of claim 20, wherein the target nucleic acid is labeled with a Cas sandwich comprising:
a randomized gRNA library complexed with a Cas sandwich protein;
the randomized gRNA library comprising a plurality of secondary gRNAs;
the secondary gRNAs comprising a tail complexed to the Cas sandwich protein and are labeled.

22. The system of claim 21, wherein the system is configured as a multiplex assay for detecting multiple target nucleotides.

23. The system of claim 21, wherein the plurality of the secondary gRNAs are fluorescently labeled.

24. The system of claim 21, wherein the plurality of secondary gRNAs is modified by addition of haptens, biotin, fluorescent labels, quantum dots, nanoparticles, enzymes, or enzyme substrates.

25. A method of performing a multiplex assay comprising:
immobilizing a gRNA to an immobilization surface;
complexing a Cas enzyme to the gRNA;
adding a target nucleic acid;
labeling the target nucleic acid;
detecting the target nucleic acid without amplification;
determining one or more results based on the detected target nucleic acid via a reader; and wherein:
the target nucleic acid is labeled with a Cas sandwich comprising:
a randomized gRNA library complexed with a Cas sandwich protein;
the randomized gRNA library comprising a plurality of secondary gRNAs;
the secondary gRNAs comprising a tail complexed to a Cas sandwich protein and are labeled; and
the randomized gRNA library comprises secondary gRNAs comprising a sequence at least 80% identical to SEQ. ID NO. 1, SEQ. ID NO. 2, or SEQ ID. NO. 3.

26. The method of claim 25, wherein the SEQ ID. NO 3 comprises a sequence of TTTV prior to a scaffold sequence.

27. A method comprising:
producing a library of gRNAs comprising a sequence at least 80% identical to SEQ. ID NO. 1, SEQ. ID NO. 2, or SEQ ID. NO. 3.

28. The method of claim 27, wherein the SEQ ID. NO 3 comprises a sequence of TTTV prior to a scaffold sequence.

29. A method comprising:
using a library of gRNAs for nucleotide detection comprising a sequence at least 80% identical to SEQ. ID NO. 1, SEQ. ID NO. 2, or SEQ ID. NO. 3.

30. The method of claim 29, wherein the SEQ ID. NO 3 comprises a sequence of TTTV prior to a scaffold sequence.

31. A gRNA library comprising a sequence at least 80% identical to SEQ. ID NO. 1, SEQ. ID NO. 2, or SEQ ID. NO. 3.

32. The gRNA library of claim 31, wherein the SEQ ID. NO 3 comprises a sequence of TTTV prior to a scaffold sequence.


SEQUENCE LISTING (3 sequences)

SEQ ID NO: 1 - RNA, 59 nt, synthetic construct
  source 1..59: mol_type = other RNA
  ncRNA 1..59: ncRNA_class = guide_RNA
caagtaaacccctaccaactggtcggggtttgaaachhhhhhhhhddddddvvvvbhbh

SEQ ID NO: 2 - RNA, 59 nt, synthetic construct
  source 1..59: mol_type = other RNA
  ncRNA 1..59: ncRNA_class = guide_RNA
caagtaaacccctaccaactggtcggggtttgaaachbhhhhbhhhhhhhbbbbbvvvv

SEQ ID NO: 3 - RNA, 43 nt, synthetic construct
  source 1..43: mol_type = other RNA
  ncRNA 1..43: ncRNA_class = guide_RNA
taatttctactaagtgtagathnhnhnhnnnnnnnnnnnnnnn